(19) United States
(12) Patent Application Publication
(10) Pub. No.: US 2009/0143639 A1
(43) Pub. Date: Jun. 4, 2009

(54) TRANS-DOUGLAS ENDOSCOPICAL SURGICAL DEVICE (TED) AND METHODS THEREOF

(76) Inventor: Michael STARK, Berlin (DE)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/352,118

(22) Filed: Jan. 12, 2009

Related U.S. Application Data
(63) Continuation-in-part of application No. PCT/IL2007/000821, filed on Jul. 2, 2007.
(60) Provisional application No. 60/830,356, filed on Jul. 13, 2006.

(51) Int. Cl. A61B 1/307; A61B 1/005

(52) U.S. Cl. 600/102; 600/135; 600/146; 600/131; 600/118

(57) ABSTRACT

The present invention discloses means and methods for providing trans-Douglas endoscopic surgical procedures in the pelvis and in other body portions, and especially gynecological, cardiological, urological, vascular, neurological or surgical operations; said method comprising steps of opening the Pouch of Douglas; inserting an U-shapeable or S-shaped endoscopic surgical device into the vagina in parallel to the blood vessels; introducing the distal end into the abdominal cavity; U-shaping or S-shaping the tubular assembly; and carrying out an operation in the pelvis.

### FIELD OF THE INVENTION

The present invention relates to a method of treating human patients by trans-Douglas endoscopic surgical procedures and to a trans-Douglas endoscopic surgical device.

### BACKGROUND OF THE INVENTION

Abdominal operations have been successfully performed since the beginning of the 19th century, firstly longitudinally and at the end of the century, transversally, Endoscopical surgery was developed during the 20^{th} century and most abdominal operations can now be performed using endoscopes.

Endoscopoic procedures are recognized to have considerable advantages over open operations (laparotomies) because of the avoidance of large incisions and the discomfort connected with them, long and expensive hospital stay, and extended period of incapacity caused by such incisions. Laparoscopic procedures are performed by a few small penetrations into the body, which cause less discomfort, and reduce the time and expense of the hospital stay and the patient's period of incapacity.

Current methods of laparoscopy require firstly filling the peritoneal cavity with carbon dioxide gas via an inflation device and maintaining inflation pressure. The gas expands the prentoneal cavity, which provides room in which surgical instruments are introduced into the selected surgical site. In parallel, the surgeon uses a variety of instruments to conduct the surgical procedures.

The number of required instruments and their repeated insertion and removal increases the time required for the procedure and its cost. It has been recognized that reduction in the time required for the procedure is desirable to reduce the length of anaesthetic required and to enable more 25 procedures to be performed in a given time.

Endoscopic devices for use in medical procedures are passed through a working channel positioned in the body cavity in order to reach an operative site next to the distal end of the endoscope. For the purposes of this description, “distal” refers to the end extending into a body and “proximal” refers to the end extending out of the body. The size of a distal assembly on the distal end of the endoscopic device, such as forceps and surgical scissors, is limited by the diameter of the endoscope's working channel.

References illustrative of the state of art are U.S. Pat. Nos. 4,759,348; 5,037,433; 5,190,541; 5,318,589; 5,562,703; 5,906,621; 6,685,724; 6,960,210; and 7,169,167.

An endoscopic surgical device, performing minimally invasive endoscopic intra-abdominal procedures with use of one entry and especially such a device, adapted to directly translate the surgeon's hands movement and orientation, as if his hands were inside the abdomen, is still a long felt need.

### SUMMARY OF THE INVENTION

It is one object of the present invention to disclose a method of providing trans-Douglas endoscopic surgical procedures in the pelvis and in other body portions, and especially gynecological, cardiological, urological, vascular, neurological or surgical operations; said method comprising steps of: a. opening the Pouch of Douglas; b. inserting an U-shapeable endoscopic surgical device into the vagina; c. introducing the distal end into the abdominal cavity in parallel to major blood vessels; d. U-shaping the tubular assembly; and, e. carrying out an operation in the pelvis.

It is another object of the present invention to disclose the method as defined above for performing minimally invasive endoscopic intraabdominal procedures; said method further comprising steps of (I) providing an endoscopic surgical device being an elongated member and (II) providing said endoscopic surgical device with a distal portion located within said body cavity, comprising operating modules, and a proximal portion comprising controlling means handled by the surgeon, located outside said body cavity, and a middle portion, adapted to the pelvic shape, interconnecting said distal portion with said proximal portion; wherein said method further comprising steps of: a. providing said distal portion comprising either (i) one operating module (effecter), or (ii) two or more effecters, adapted to directly translate the surgeon's hands movement and orientation, as they interact with the tools; providing said effecters with means adapted to allow a single instrument to carry out procedures which normally would require both hands of the surgeon and/or two invasive endoscopic devices; providing said distal portion with auxiliary means, and selecting said auxiliary means from a group consisting of scopes or other image and data acquiring means, channels for tools, effecters and fluids inlets and/or outlet, lighting means, radiofrequency (RF), laser or ultrasound emitting means, sensors, diagnostic tools, OCT-diagnostic tools, optics, or any combination thereof; b. providing said proximal portion comprising controlling means adapted to maneuver and operate at least a portion of said effecters and auxiliary means; and, c. providing said middle portion interconnects said proximal portion with said distal portion; suitable to provide a predetermined shape, especially a shape selected from a shape adapted to the pelvic anatomy, namely U-like shape, S-like shape, partially linear shape and partially non-linear shape; said middle portion is preferably characterized by a tubular or polygonal cross section, and comprises a plurality of channels exceeded from the proximal end to the distal end.

It is another object of the present invention to disclose the method as defined above; wherein the method further comprising a step of providing said middle portion with either S-like or U-like shapes, adapted to the contour of the pelvis of the patient.

It is another object of the present invention to disclose the method as defined above; wherein the method further comprising a step of providing said device with an optic system, adapted to provide either 2D or 3D vision at or adjacent its distal end.

It is another object of the present invention to disclose the method as defined above; wherein the method further comprising a step of providing said device configuration switching mechanism to either reversibly or irreversibly switching between two or more configurations, especially from initial configuration of approximately linear shape to either U-like or S-like shapes, and vice versa, from initial either U-like or S-like shapes to an approximated linear shape.

It is another object of the present invention to disclose the method as defined above; wherein the method further comprising a step of providing said device with configuration switching mechanism which is operated mechanically, hydraulically, electrically or by a combination of the same; said switching mechanism is possibly operated by either wired or wireless (remote controlled) means.

It is another object of the present invention to disclose the method as defined above; wherein the method especially useful for teaching and training purposes and/or operating multiple tasks simultaneously, wherein said proximal portion comprises a plurality of N sets of control handles, N is an integer number equal to or higher than 1, especially wherein N equals 2.

It is another object of the present invention to disclose the method as defined above; wherein the method further comprising a step of selecting at least a portion of said effecters from a group consisting of scissors, forceps, harmonic knife, monopolar knife, clips, blades, RF, cryotherapy, laser, monopolar knife and/or bipolar coagulation, ultrasound, spillage and suction modules, optics, especially optical means for 2D or 3D sight, light sources, loops and snare-like members, basket-like members, cleansing modules, and optical cleansing means.

It is another object of the present invention to disclose the method as defined above; wherein the method further comprising a step of selecting said optics from a group consisting of one or more either 2D or 3D scopes, one or more optic fibers, array of optic fibers, binocular, optical paths, imaging means, and especially OCT, ultrasound probes adapted for detection of blood vessels, tool for diagnostics or any combination of the same.

It is another object of the present invention to disclose the method as defined above; wherein the method further comprising a step of providing the device with optical cleansing modules, and providing the device with means for applying fluids, especially saline towards the optic tip portion located at the distal portion of the device; and especially wherein said cleansing modules and optical cleansing means are applied as high pressure water sprinkles directed to at least one scope distal tip.

It is another object of the present invention to disclose the method as defined above; wherein the method further comprising a step of providing said cleansing modules with wiper adapted to wipe the optic tip.

It is another object of the present invention to disclose the method as defined above; wherein the method further comprising a step of operating at least a portion of said effecters by electrically, hydraulically, mechanically, robotically, or by any combination of those techniques; and manipulating said electrical operated effecters by either wired or wireless (remote controlled) means.

It is another object of the present invention to disclose the method as defined above; wherein the method further comprising a step of providing the device by a main longitudinal X axis; maneuvering at least a portion of said effecters along one or more of the X, Y, Z axes or any combination thereof, especially along the XY, XZ, YZ planes or any combination of those planes, such that one or more of said effecters are adapted to reciprocate along one or more of said axes, rotate around one or more of said axes, retract or otherwise expand or protract along one or more of said axes, maneuver upwards of downwards along one or more of said axes, set the effecters in their ON or OFF; Activated or Deactivated; Open (e.g., widen), gradually Close or completely Close (e.g., tighten); Loose or Secured modes or any combination thereof.

It is another object of the present invention to disclose the method as defined above; wherein the method further comprising a step of providing said device with a length of the device is about 40 to about 60 cm, especially 50 cm; the outer width is between about 20 to about 50 mm, especially 25, 35 or 40 mm; the number of internal channels is between about 1 to about 10, especially 4, wherein 1^{st} channel is of about 5 to about 15 mm, especially 10 mm, useful for needles and suturing materials, or morcellators, 2^{nd} and 3^{rd} channels are of about 20 2 to about 10 mm, especially about 5 mm, useful for various instruments and fluids flow, and 4^{th} channel is of about 1 to about 5 mm, especially about 3 mm useful for fluids flow, especially about for spillage and suction.

It is another object of the present invention to disclose the method as defined above; wherein the method further comprising a step of providing said device by biocompatible building materials; and selecting at said materials from a group consisting of metals, especially stainless steel, polymers, shape memory alloys, especially nitinol, electrocative polymers, glassware, composite materials, cardboard or any mixture thereof.

It is another object of the present invention to disclose the method as defined above; wherein the method further comprising a step of providing said proximal portion to be at least temporarily immobilized to a fixation, especially wherein said fixation is selected from the operation table or a moveable tripod.

It is another object of the present invention to disclose a method of treating human body by the method as defined above; wherein said treatment is useful for applications selected from a group consisting (i) urological applications, especially nephrectomy, excision of ureteric and/or bladder stones, bladder tumors, kidney stones, kidney tumors, operation of the ureter, excisions biopsies and treatment thereof, treatment of stress incontinence through suturing, slings and/or mashes; (ii) surgical application, especially cholecystectomy, liver biopsies, gastric tumors splenectomy, gastrointestinal tumors (cholectomies), appendectomy, tumors of the pancreas (carcinoma, or insulinoma), or lymphydenectomy; (iii) endocrinological applications, especially suprarenal tumors or insulinoma; (iv) gynecological applications: TED assisted hysterectomy (supracervical or total), TED total hysterectomy, 10 myomectomies, or treatment of fibroids with RF, cryotherapy, occlusion of blood vessels, excision of extrauterine pregmensies, tubal surgery; (v) cardiovascular applications, especially diagnostic or surgical, implantation of heart valves and grafts of or to the major blood vessels, operations on blood vessels, like the A. Renalis; and (vi) neurological applications: application of electrodes to nerves arising from the Sacral and Lumbar Plexi.

It is another object of the present invention to disclose a method of providing trans-Douglas endoscopic surgical procedures in the abdominal cavity and other body portions, especially in the gallbladder, blood vessels, nerves, liver, pancreas, spleen, kidney, colon, jejunum, or ileum, heart, nerve or combination thereof; said method comprising steps of: opening the Pouch of Douglas; inserting an S-shapeable endoscopic surgical device into the vagina; possibly after insertion of a sleeve, introducing the distal end into the abdominal cavity, S-shaping the tubular assembly, and carrying out an operation in the abdominal cavity.

It is another object of the present invention to disclose the method as defined above; wherein the method is utilized for performing minimally invasive endoscopic intraabdominal procedures; said method further comprising steps of (I) providing an endoscopic surgical device being an elongated member and (II) providing said endoscopic surgical device with a distal portion located within said body cavity, comprising operating modules, and a proximal portion comprising controlling means handled by the surgeon, located outside said body cavity, and a middle portion, adapted to the pelvic shape, interconnecting said distal portion with said proximal portion; wherein said method further comprising steps of: a providing said distal portion comprising either (i) one operating module (effecter), or (ii) two or more effecters, adapted to directly translate the surgeon's hands movement and orientation, as they interact with the tools; providing said effecters with means adapted to allow a single instrument to carry out procedures which normally would require both hands of the surgeon and/or two invasive endoscopic devices; providing said distal portion with auxiliary means, and selecting said auxiliary means from a group consisting of scopes or other image and data acquiring means, channels for tools, effecters and fluids inlets and/or outlet, lighting means, radiofrequency (RF), laser or ultrasound emitting means, sensors, diagnostic tools, OCT-diagnostic tools, optics, or any combination thereof; b. providing said proximal portion comprising controlling means adapted to maneuver and operate at least a portion of said effecters and auxiliary means; and, c. providing said middle portion interconnects said proximal portion with said distal portion; suitable to provide a predetermined shape, especially a shape selected from a shape adapted to the pelvic anatomy, namely U-like shape. S-like shape, partially linear shape and partially nonlinear shape; said middle portion is preferably characterized by a tubular or polygonal cross section, and comprises a plurality of channels exceeded from the proximal end to the distal end.

It is another object of the present invention to disclose the method as defined above; wherein the method further comprising a step of providing said device with configuration switching mechanism which is operated mechanically, hydraulically, electrically or by a combination of the same; said switching mechanism is possibly operated by either wired or wireless (remote controlled) means.

It is another object of the present invention to disclose the method as defined above; wherein the method further comprising a step of providing said middle portion with either S-like or U-like shapes, adapted to the contour of the pelvis of the patient.

It is another object of the present invention to disclose an endoscopic surgical device for use in performing minimally invasive endoscopic intraabdominal procedures; said device is an elongated member and characterized by a distal portion located within said body cavity, comprising operating modules, and a proximal portion comprising controlling means handled by the surgeon, located outside said body cavity, and a middle portion, adapted to the pelvic shape, interconnecting said distal portion with said proximal portion; wherein a. said distal portion comprising either (i) one operating module (effecter), or (ii) two or more effecters, adapted to directly translate the surgeon's hands movement and orientation, as they interact with the tools; said effecters allow a single instrument to carry out procedures which normally would require both hands of the surgeon and/or two invasive endoscopic devices; said distal portion further comprising auxiliary means, selected from a group consisting of scopes or other image and data acquiring means, channels for tools, effecters and fluids inlets and/or outlet, lighting means, radiofrequency (RF), laser or ultrasound emitting means, sensors, diagnostic tools, OCT-diagnostic tools, optics, or any combination thereof; b. said proximal portion comprising controlling means adapted to maneuver and operate at least a portion of said effecters and auxiliary means; c. said middle portion interconnects said proximal portion with said distal portion; suitable to provide a predetermined shape, especially a shape selected from a shape adapted to the pelvic anatomy, namely U-like shape, S-like shape, partially linear shape and partially non-linear shape; said middle portion is preferably characterized by a tubular or polygonal cross section, and comprises a plurality of channels exceeded from the proximal end to the distal end.

It is another object of the present invention to disclose the aforesaid device, wherein said middle portion is characterized by either S-like or U-like shapes, adapted to the contour of the pelvis of the patient.

It is another object of the present invention to disclose the aforesaid device wherein said middle portion is adapted to either reversibly or irreversibly switch between two or more configurations, especially from initial configuration of approximately linear shape to either U-like or S-like shapes, and vice versa, from initial either U-like or S-like shapes to an approximated linear shape.

It is another object of the present invention to disclose the aforesaid device wherein said configuration switching mechanism is operated mechanically, hydraulically, electrically or by a combination of the same; said switching mechanism is possibly operated by either wired or wireless (remote controlled) means.

### BRIEF DESCRIPTION OF THE DRAWINGS

These and other features and advantages of the present invention will be more fully disclosed in, or rendered obvious by, the following detailed description of the preferred embodiments of the invention, which are to be considered together with the accompanying drawings wherein like numbers refer to like parts and further wherein:

FIG. 1 shows a functional diagram of the trans-Douglas endoscopic device;

FIG. 2a and FIG. 2b are elevational views of the U- and S-shaped tubular assemblies with one bendable tubular member;

FIG. 2c is a cross-sectional view of the rigid member of the tubular assembly;

FIG. 3a and FIG. 3b are elevational views of the U- and S-shaped tubular assemblies with two joint members;

FIG. 4a and FIG. 4b are isometric views of the distal end of the tubular assembly before insertion into a female patient's body and in operative field (not shown), respectively;

FIG. 5 shows a flow chart of the method for use of the endoscopic surgical device in the case of S-shaped tubular assembly;

FIG. 6 shows a flow chart of the method for use of the endoscopic surgical device in the case of the U-shaped tubular assembly;

FIG. 7 is a schematic view of the U-shaped tubular assembly in anatomic cavity;

FIG. 8 is a schematic view of the S-shaped tubular assembly in anatomic cavity; and,

FIG. 9 is a schematic view of a sleeve useful to accommodates the device as defined above.

### DETAILED DESCRIPTION OF THE INVENTION

The present invention generally pertains to methods of treating human (and other female mammalians) patients by trans-Douglas endoscopic surgical procedures and to a trans-Douglas endoscopic surgical device. The trans-Douglas endoscopic device in accordance with the present invention is preferably utilized in the pelvis and in the abdominal cavity. The device is inserted into operative field through the pouch of Douglas.

The term ‘pouch of Douglas’ refers hereinafter to the extension of the peritoneal cavity between the rectum and back wall of the uterus in the female human body. It is also known by the names Pouch of Douglas; Douglas space, Douglas cul-de-sac, rectouterine pouch, and rectouterine excavation.

The term ‘about’ refers hereinafter to a value being 20% more or less than the defined measure.

All women have easy access to the abdominal cavity through the Pouch of Douglas. This is a peritoneal fold between the sacro-uterine ligaments which is easily reachable and opened (dissected) under vision.

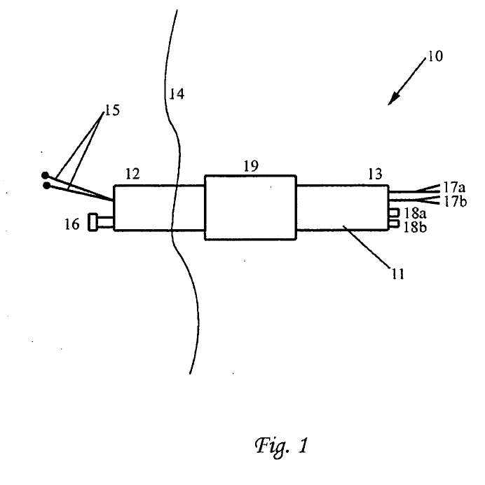

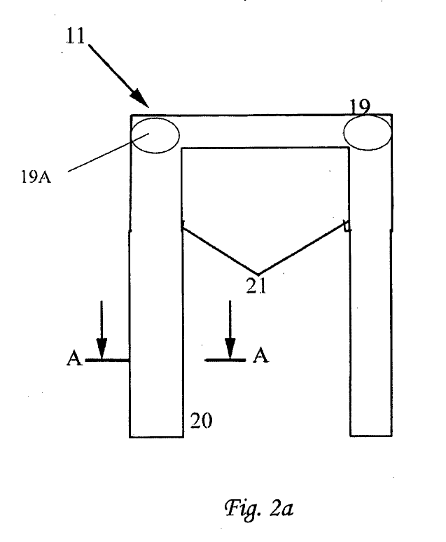

A schematic illustration of a trans-Douglas endoscopic device 10 in working position is shown in FIG. 1. The device 10 comprises a tubular assembly 11 having a proximal end 12 and a distal end 13. The proximal end 12 refers to the end extending out of the body and the distal end 13 refers to the end extending into the body. The parts of the device 10, located on the left of body boundary 14 are disposed externally and the parts of the device 10 located on the right of the 5 boundary 14 are disposed internally. Specifically, control handles 15 and an ocular 16 for observation of operative field are disposed externally at the proximal end 12, whereas forceps 17a, scissors 17b, a light source 18a, and a front lens 18b-internally at the distal end 13. A mechanical drive 19 for shape change of the tubular assembly 11 is in the tubular assembly 11, 19a are joints or axes of the device.

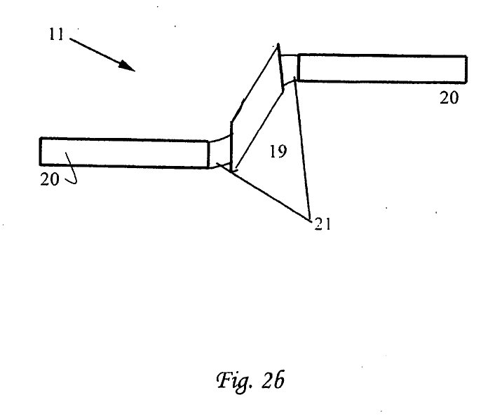

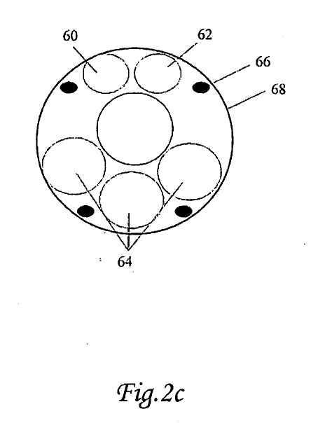

Referring to FIGS. 2a and 2b, the tubular assembly 11 according to one of embodiments of the invention comprises at least two rigid tubular members 20 and one bendable member 21. FIG. 2a and FIG. 2b are schematic views of U- and S-shaped tubular assemblies 11.

FIG. 2c is a cross-sectional view of the rigid member 20 of the tubular assembly 11 in the plane of cross-section A-A shown in FIG. 2a. In accordance with one embodiment of the invention two optical fibers 60 and 62 and slidable shafts 64 are housed in a tube 68.

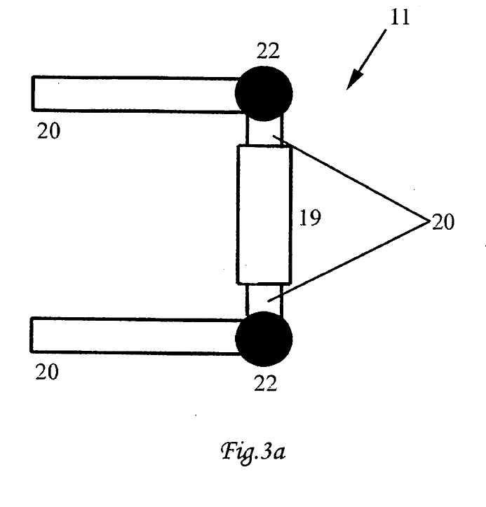

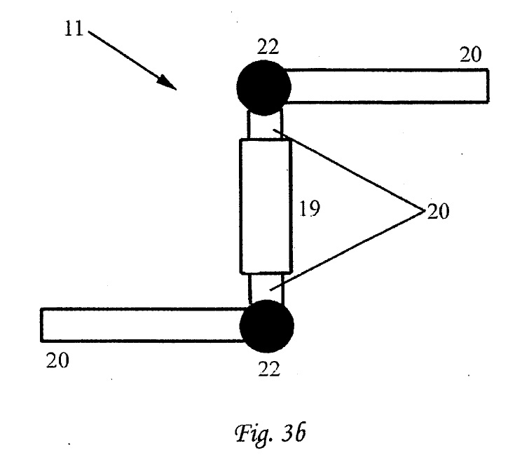

Referring to FIGS. 3a and 3b, the tubular assembly 11 according to other embodiment of the invention comprises at least three rigid tubular members 20 and two joint members 22. FIG. 3a and FIG. 3b are schematic views of U-like and S-like shaped tubular assemblies 11.

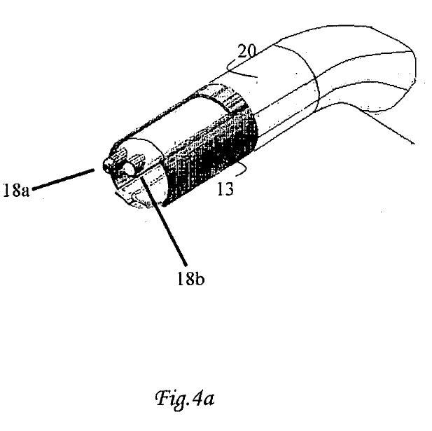

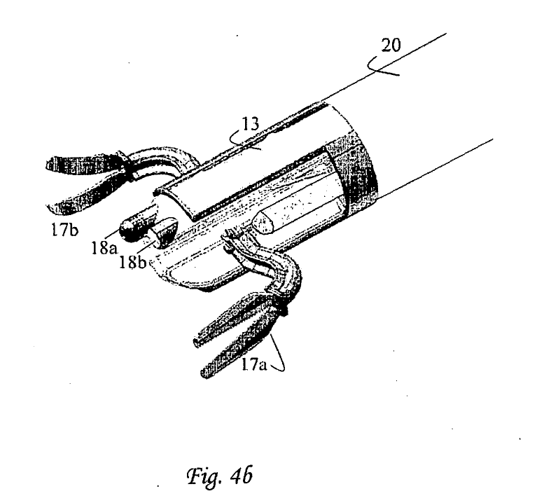

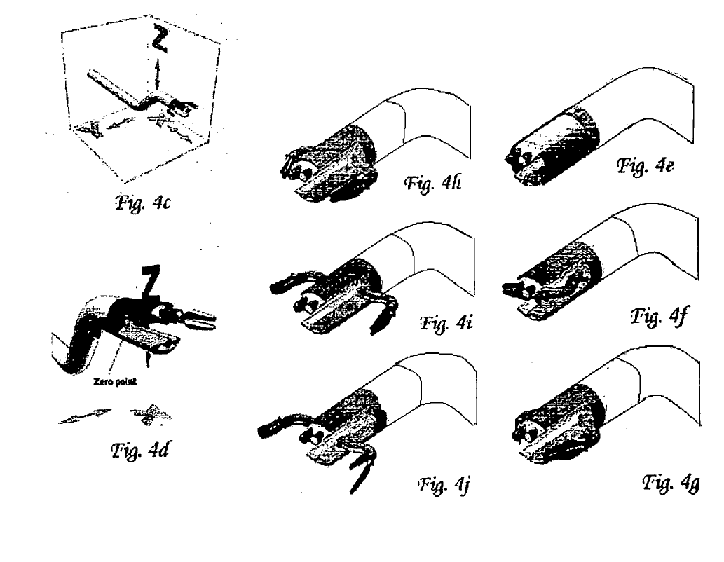

FIG. 4a to 4j illustrate the distal end 13 before insertion into female patient's body and in 20 operative field (not shown), respectively. In the process of device insertion (FIG. 4a) the forceps 17a and the scissors 17b are located in the distal end 13 while the light source 18a and front lens 18b enable to perform video observation of insertion process. Upon reaching the operative field, forceps 17a and scissors 17b come forward and are ready to perform an operation.

FIG. 4b illustrates a top and front view of a chelating tool according to one embodiment of the invention, comprising two or more opposing operating modules (effecters), here, e.g., forceps (17a) and scissors (17b) adapted to directly translate the surgeon's hands movement and orientation, as they interact with the tools. The effecters are set in a ‘lobster’-like arrangement, which allows a single instrument to carry out procedures which normally would require both hands of the surgeon. Hence, only one opening in the body (here, the vagina) is required to introduce two opposite operating modules.

It is in the scope of the invention wherein the effecters are selected in a non-limiting manner from a group consisting inter alia of scissors, forceps, monopolar knife, clips, blades, spillage and suction modules, optics, especially optical means for 2D or 3D sight, light sources, loops and snare-like members, basket-like members, cleansing modules, and optical cleansing means. The optical cleansing means is provided, in a non-limiting manner, from means for applying fluids (especially saline towards the optic tip portion. High pressure water sprinkles directed to the scope tip is an example of such a cleansing module. The effecters are operated, according to one embodiment of the invention, electrically, mechanically, hydraulically, robotically, either wire or wireless (remote controlled) operated, or by any combination of those techniques.

According to one embodiment of the invention, the aforesaid tool is utilized in an either S-shape or U-like shape endoscope, as presented in FIG. 4c. The endoscopic surgical device is characterized by a main longitudinal axis, i.e., along axis X, wherein the effecters, located at the distal portion of the device, are adapted to maneuver along one or more of the X, Y, Z axes or any combination thereof, e.g., along the XY, XZ, YZ planes or any combination of those planes. Hence for example as pointed out in FIG. 4d, one or more of the effecters is adapted in a non-limiting manner to reciprocate back and force along axis X, rotate around said axis, retract or otherwise expand or protract along the Y axis, maneuver upwards of downwards along axis Z, set the effecters in; their ON or OFF; Activated or Deactivated; Open (e.g., widen), gradually Close, or completely Close (e.g., tighten); Loose or Secured modes or any combination thereof.

It is also in the scope of the invention wherein the effecters are interconnected to at least one mutual anchoring point (zero point). This zero point is a useful reference point in precise operation of at least one or all of the effecters.

In the initial steps of operation, the effecters are adapted to be securably accommodated within the inner bore of the device, such that its external diameter is small and no protruding members are provided (See FIG. 4e). After inserting the distal portion of the device, via the vaginal walls, inside the body of the patient, the effecters are adapted to controllably escape from their initial ‘shrinked’ configuration (See FIG. 4f). The 2D or 3D orientation of each of the effecters, as well as the mutual orientation of the two or more effecters is illustrated in a non-limiting manner in FIGS. 4g to 4i, and the either separate or simultaneous operation of the effecters is depicted in FIG. 4j, here opening the scissors.

According to yet another embodiment, at least a portion of the effecters is adapted to face each other, i.e., wherein the effecter is approximately perpendicular to the main longitudinal axis X, to be parallel to axis X or to be, at least temporarily 2D- or 3D-oriented a combination of said two configurations.

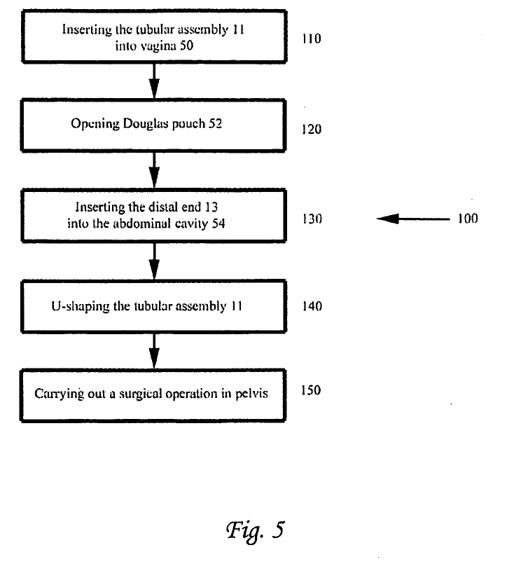

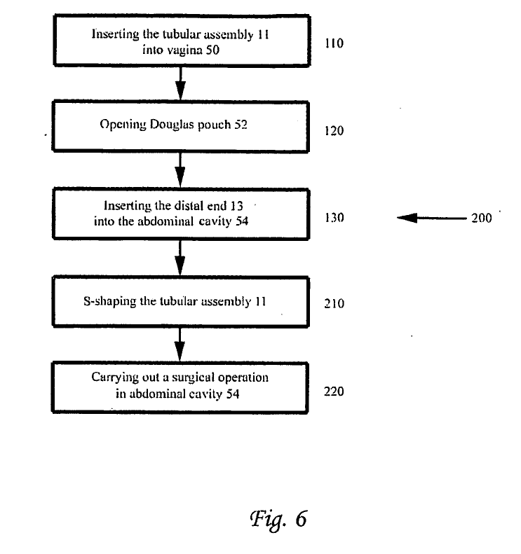

With further reference to FIGS. 5 and 6, flow charts provide step sequences of method for use the trans-Douglas endoscopic device 10 in accordance with two different embodiments of the invention. A step sequence 100 for the U-like shaped assembly 11 is shown in FIG. 5, while a step 5 sequence 200 for the S-like shaped assembly 11 is shown in FIG. 6.

In accordance with one embodiment (FIG. 5) the step sequence 100 comprises following steps inserting 110 the device into the vagina 50, opening 120 the Pouch of Douglas 52, inserting 130 the distal end 13 into the abdominal cavity 54, the U-shaping 140 the tubular assembly, and carrying Out 150 an operation in the pelvis.

In accordance with another embodiment (FIG. 6) the step sequence 200 comprises following steps: inserting 110 the device into the vagina 50, opening 120 the Pouch of Douglas 52, inserting 130 the distal end 13 into the abdominal cavity 54, the S-shaping 210 the tubular assembly, and carrying out 220 an operation in the pelvis.

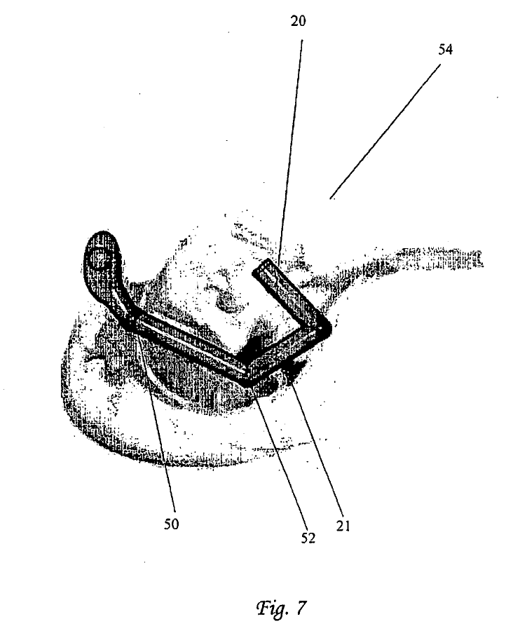

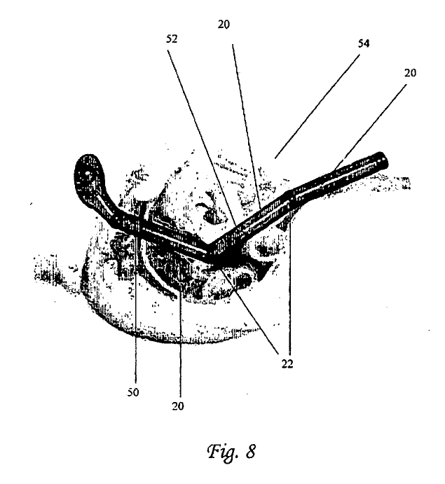

FIG. 7 illustrated the tubular assembly 11 in a working position. In accordance with one 15 embodiment the U-shaped device is shown in working position. When inserted into the vagina 50 and further passed through the Pouch of Douglas 52, the assembly 11 reaches the abdominal cavity 54. After U-shaping bendable member 21 the trans-Douglas endoscopic surgical device 10 (not shown) is ready to carry out a surgical operation in the pelvis. A working position for carrying out surgical operation in the abdominal cavity 54 is shown in FIG. 8. When passed along the same path through the vagina 50 and the Pouch of Douglas 52, the assembly 11 undergoes S-shaping in the abdominal cavity 54. In accordance with another embodiment S-shaping is implemented by means of the joint members 22 located between the rigid members 20.

In accordance with the invention the trans-Douglas endoscopic device 10 (FIG. 1) comprises the tubular assembly 11, control knobs 15 and the ocular at the proximal end 12 of the tubular assembly 11 and the forceps 17a, the scissors 17b, the light source 18a, and the front lens 18b at the distal end 13 of the tubular assembly 11. Mechanical driver 19 is disposed inside the tubular assembly 11 to perform shape change of the tubular assembly 11.

The control knobs 15, mechanically, hydraulically or electrically coupled with the forceps 17a, the scissors 17b, enable implementation of remote surgical endoscopic operations. The ocular 16 30 optically coupled with front lens 18b enables observation of the operative field, illuminated by the light source 18a. FIG. 3c shows the shafts 64 that mechanically couple control knobs 15 with the forceps 17a and scissors 17b, and the optical fibers 60 and 62 that optically couple the ocular 16 with the light source 18a and the front lens 18b. The cables 66 feed the mechanical driver 19. AU the above-mentioned parts are housed in the tube 68.

In accordance with the invention, the trans-Douglas endoscopic device 10 is adapted to be inserted into a female body through the Pouch of Douglas. At the beginning of operation the 5 device 10 is outside of a female patient's body. In primary position the distal end 13 is closed and forceps 17a and scissors 17b are inside the distal end 13 (FIG. 4a). After that an operator (not shown) inserts the tubular assembly 11 into the vagina 50 under parallel video observation by means of ocular 16 (FIGS. 7, 8) Further, an operator performs opening the Pouch of Douglas 52 and inserts the tubular assembly 11 into the abdominal cavity 54.

At the next step an operator performs shape change of the tubular assembly 11 by means of the mechanical drive 19. In accordance with one alternative embodiment of the invention, the tubular assembly 11 takes the U-like form. U-like shape is adapted for performing endoscopic surgical operation in the pelvis (e.g., cardiac, vascular, neurological, gynecological, urological, surgical operations). In accordance with another alternative embodiment of the invention, the 15 tubular assembly 11 takes the S-like form. S-shape is adapted for performing endoscopic surgical operation in the abdominal cavity 54 (such as gallbladder, liver, pancreas, spleen, kidney, colon, jejunum, ileum operations etc).

In accordance with further one alternative embodiment of the invention, the tubular assembly 11 (FIG. 2a, 2b) comprises at least two rigid tubular members 20 and at least one bendable member 20 21. The mechanical driver 19 bends the bendable member 21, and in this way the tubular assembly takes U-like (FIG. 2a) or S-like (FIG. 2b) shape.

In accordance with other alternative embodiment of the invention, the tubular assembly 11 (FIG. 3a, 3b) comprises at least three rigid tubular members 20 and at least two joint members 22. The mechanical driver 19 bends the joint members 22, and in this way the tubular assembly takes U-25 or S-like shape.

As the pouch of Douglas is used for insertion of the endoscopic device 10, a diameter of the tubular assemblage 11 can be substantially bigger (e.g., up to about 45 mm) than a diameter of traditional endoscopic device. It enables use of more surgical tools and morcellators of larger size. The entry point even enables removal of tissues at the end of the operation without using a 30 morcellator (gallbladder, fibroids etc.).

The direction of insertion of the endoscopic device 10 is parallel to major blood vessels. Additionally, manipulations with the endoscopic device 10 are implemented under visual control and low CO₂ pressure. Both these factors decrease surgical implication probability.

Evaluation shows that necessary insufflation pressure should be lower than insufflation pressure in traditional laparoscopy. Contrary to traditional endoscopical procedures, the endoscopic device 10 is introduced first, e.g., through a sleeve, and the insufflation is performed afterwards when necessary.

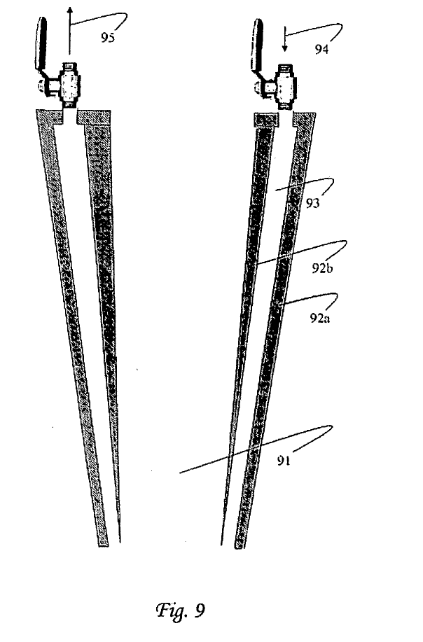

Reference is now made to FIG. 9, illustrating a cross section of a rigid or flexible elongated Hegar-like sleeve, having at least one continuous bore (91) adapted to at least reversibly accommodate the endoscopic surgical device of the present invention. According to one embodiment of the invention, the sleeve is jacked and/or comprises one or more longitudinal tunnels. Hence for example, and in a non-limiting manner, the sleeve is comprised of two conduits (e.g., 93) barriered for example by an inner wall (92a) and outer wall (92b), enabling pressurized CO₂ to be applied in one conduit via a first valve (94) while fluid drainage is allowed via a second valved conduit (95). This sleeve is possibly elongated or curved, rigid or flexible, metal-made and/or plastic-made, disposable of adapted for multiple use. The length of the sleeve is between about 15 to about 25 cm, e.g., about 17 cm; its inner width is in a measure suitable to accommodate the aforesaid endoscopic surgical device. The sleeve is either a linear or a curved member adapted to accommodate a shaped device. A sleeve will not be used in preformed devices, and in such a case the CO₂ is introduced via one of the TED-channels.

It is well in the scope of the invention wherein the endoscopic surgical device as defined in any of the above comprises optic system, adapted to provide a clear 2D or 3D vision at its distal portion, e.g., the device comprises multiple scopes, binocular, array of optic fibers or optical paths.

It is also in the scope of the invention wherein the endoscopic surgical device as defined in any of the above is adapted to reversibly or irreversibly switch between two or more configurations, e.g., from initial approximately linear member to either U-like or S-like shaped member, and vice versa. The configuration switching mechanism is provided in a non-limiting manner either mechanically, hydraulic, electrically or both.

It is in the scope of the invention wherein the distal and/or the middle portion of the device 30 comprises one or more engines adapted to actuate and/or activate the effecters and the joints of the middle portion. The engines are situated in the distal and middle portion of the device and are operated by the surgeon or his/her assistance by either a remote control (wireless) means and/or by means of a plurality of one or more communication wires exceeded from said distal end to said proximal end.

It is also in the scope of the invention wherein the endoscopic surgical device as defined in any of the above is adapted for treatment modality selected in a non-limiting manner from a group 5 consisting inter alia of RF, cryotherapy, laser, monopolar knife and/or bipolar coagulation, harmonic knife, clips, monopolar knife, ultrasound etc.

It is also in the scope of the invention wherein the endoscopic surgical device as defined in any of the above is adapted to enable diagnostic modality selected in a non-limiting manner from a group consisting inter alia of optical, ultrasound, ultralight OCT, biopsy, especially traditional biopsy and proteomics, is also in the scope of the invention wherein the endoscopic surgical device as defined in any of the above comprises a plurality of N sets of control handles (15), N is an integer number equal to or higher than 1, e.g., N equals 2. In this embodiment, two or more surgeons or assistants can operate control handles in the proximal portion of the device in order to maneuver one or more effecters located at its distal portion. Such as embodiment is especially useful for teaching and training purposes. It is also in the scope of this embodiment wherein multiple tasks are operated simultaneously. Hence for example, diagnostic procedures (e.g., sampling the targeted tissue) are performed at the same time that other surgical processes are applied.

It is also in the scope of the invention wherein the at least a portion of the effecters as defined in any of the above are operated manually or robotically by e.g., by either wired or wireless controlling means.

It is also in the scope of the invention wherein the endoscopic surgical device as defined in any of the above is adapted to be sterilized or otherwise cleansed & decontaminated, such that reusable operation is provided. Alternatively or additionally, the aforesaid device is potentially provided as a disposable endoscopic surgical device. It is acknowledged in this respect that according to another embodiment, only the envelope is adapted to be disposable.

It is also in the scope of the invention wherein the endoscopic surgical device as defined in any of the above is characterized by a three conceptual portions, namely proximal portion whereat the control panel (outside the body of the patient), knobs and operating mechanism is located: middle portion, usually characterized by an S-like or U-like shaped elongated (e.g., tubular or polygonal cross section) neck, suitable to contain at least one operating tunnel, and/or a plurality of open bores parallel to its main longitudinal axis; and distal portion (inside the body of the patient), whereat the effecters are located.

According to one specific embodiment of the invention, provided herein as an example, the length of the device is about 40 to about 60 cm, e.g., 50 cm; the outer width is between about 20 to about 50 mm, e.g., 25, 35 or 40 mm: the number of internal channels is between about 1 to 5 about 10, especially 4, wherein 1^{st} channel is of about 5 to about 15 mm, e.g., 10 mm, useful for needles and suturing materials, morcellators etc, 2^{nd} and 3^{rd} channels are of about 2 to about 10 mm, e.g., 5 mm, useful for various instruments and fluids flow, and 4^{th} channel is of about 1 to about 5 mm, e.g., 3 mm, useful for fluids flow, e.g., for spillage and suction. Additionally or alternatively, two lateral channels are applied, each of the two channels is of about 15 to about 10 35 mm, e.g., 25 mm. Accordingly, multiple effecters are provided in the distal portion of the device, e.g., scissors (on the right) and forceps (on the left). The device is at least partially made of materials selected in a non-limiting manner from a group consisting of metals, especially stainless steel, polymers, shape memory alloys, such as nitinol, electrocative polymers, glassware, composite materials, cardboard or any mixture thereof.

It is acknowledged in this respect that at least one monitor, intercommunicated with the device's scope, is introduced in front of the operator, e.g., above and adjacent to the patient, such that the surgeon's head is positioned frontwise, and easy operation is provided.

According to one embodiment of the invention, the aforesaid proximal portion is at least temporarily immobilized. e.g., to the operation table, patient's bed, moveable tripod etc. Hence, the neck comprises, inter alia, connecting means, e.g., ring-like connector, adapted to interconnect any commercially available immobilizing means.

It is also in the scope of the invention wherein the endoscopic surgical device as defined in any of the above is especially adapted for applications selected in a non-limited manner from a group consisting of (i) urological applications, especially nephrectomy, excision of ureteric and/or bladder stones, bladder tumors, kidney stones, kidney tumors, operation of the ureter, excisions biopsies and treatment thereof, treatment of stress incontinence through suturing, slings and/or mashes; (ii) surgical application, especially cholecystectomy, liver biopsies, gastric tumors, splenectomy, gastrointestinal tumors (like cholectomies), appendectomy, tumors of the pancreas (carcinoma, insulinoma etc), or lymphydenectomy; (iii) endocrinological applications, 30 especially suprarenal tumors or insulinoma; (iv) gynecological applications: TED assisted diskectomy (supracervical or total), hysterectomy, TED total hysterectomy, myomectomies, or treatment of fibroids with RF, cryotherapy, occlusion of blood vessels excision of extrauterine pregmensies, tubal surgery etc; (v) cardiovascular applications, especially diagnostic or surgical, implantation of heart valves and grafts of the major blood vessels, operations on blood vessels, like the A. Renalis etc; (vi) neurological applications: application of electrodes to nerves arising from the Sacral and Lumnbar Plexi.

It is also in the scope of the invention wherein the endoscopic surgical device as defined in any of the above is applied in a combined procedure involving inter alia laparoscopic methods and transgastric methods.

According to one embodiment of invention, a method of utilizing trans-Douglas endoscopic surgical procedures in the pelvis and in other body portions, and especially gynecological, cardiological, urological, vascular, neurological or surgical operations for treating human or other female mammalian patients is disclosed. The method comprises steps of: a. opening the Pouch of Douglas; b. inserting an U-shapeable endoscopic surgical device into the vagina; c. introducing the distal end into the abdominal cavity in parallel to major blood vessels; d. U-shaping the tubular assembly; and, e. carrying out an operation in the pelvis.

According to another embodiment of invention, the aforesaid method further comprises steps of (I) providing an endoscopic surgical device being an elongated member and (II) providing said endoscopic surgical device with a distal portion located within said body cavity, comprising operating modules, and a proximal portion comprising controlling means handled by the surgeon, located outside said body cavity, and a middle portion, adapted to the pelvic shape, interconnecting said distal portion with said proximal portion; wherein said method further comprising steps of a. providing said distal portion comprising either (i) one operating module (effecter), or (ii) two or more effecters, adapted to directly translate the surgeon's hands movement and orientation, as they interact with the tools; providing said effecters with means adapted to allow a single instrument to carry out procedures which normally would require both hands of the surgeon and/or two invasive endoscopic devices; providing said distal portion with auxiliary means, and selecting said auxiliary means from a group consisting of scopes or other image and data acquiring means, channels for tools, effecters and fluids inlets and/or outlet, lighting means, radiofrequency (RF), laser or ultrasound emitting means, sensors, diagnostic tools, OCT-diagnostic tools, optics, or any combination thereof; b. providing said proximal portion comprising controlling means adapted to maneuver and operate at least a portion of said effecters and auxiliary means; and, c. providing said middle portion interconnects said proximal portion with said distal portion; suitable to provide a predetermined shape, especially a shape selected from a shape adapted to the pelvic anatomy, namely U-like shape. S-like shape, partially linear shape and partially non-linear shape; said middle portion is preferably characterized by a tubular or polygonal cross section, and comprises a plurality of channels exceeded from the proximal end to the distal end.

According to another embodiment of invention, the aforesaid method further comprises a step of providing said middle portion with either S-like or U-like shapes, adapted to the contour of the pelvis of the patient.

According to another embodiment of invention, the aforesaid method further comprises a step of providing said device with an optic system, adapted to provide either 2D or 3D vision at or adjacent its distal end.

According to another embodiment of invention, the aforesaid method further comprises a step of providing said device configuration switching mechanism to either reversibly or irreversibly switching between two or more configurations, especially from initial configuration of approximately linear shape to either U-like or S-like shapes, and vice versa, from initial either U-like or S-like shapes to an approximated linear shape.

According to another embodiment of invention, the aforesaid method further comprises a step of providing said device with configuration switching mechanism which is operated mechanically, hydraulically, electrically or by a combination of the same; said switching mechanism is possibly operated by either wired or wireless (remote controlled) means.

According to another embodiment of invention, the aforesaid method is especially useful for teaching and training purposes and/or operating multiple tasks simultaneously, wherein said proximal portion comprises a plurality of N sets of control handles, N is an integer number equal to or higher than 1, especially wherein N equals 2.

According to another embodiment of invention, the aforesaid method further comprises a step of selecting at least a portion of said effecters from a group consisting of scissors, forceps, harmonic knife, monopolar knife, clips, blades, RF, cryotherapy, laser, monopolar knife and/or bipolar coagulation, ultrasound, spillage and suction modules, optics, especially optical means for 2D or 3D sight, light sources, loops and snare-like members, basket-like members, cleansing modules, and optical cleansing means.

According to another embodiment of invention, the aforesaid method further comprises a step of selecting said optics from a group consisting of one or more either 2D or 3D scopes, one or more optic fibers, array of optic fibers, binocular, optical paths, imaging means, and especially OCT, ultrasound probes adapted for detection of blood vessels, tool for diagnostics or any combination of the same.

According to another embodiment of invention, the aforesaid method further comprises a step of providing the device with optical cleansing modules, and providing the device with means for applying fluids, especially saline towards the optic tip portion located at the distal portion of the device; and especially wherein said cleansing modules and optical cleansing means are applied as high pressure water sprinkles directed to at least one scope distal tip.

It is another object of the present invention to disclose the method as defined above; wherein the method further comprising a step of providing said cleansing modules with wiper adapted to wipe the optic tip.

According to another embodiment of invention, the aforesaid method further comprises a step of operating at least a portion of said effecters by electrically, hydraulically, mechanically, robotically, or by any combination of those techniques; and manipulating said electrical operated effecters by either wired or wireless (remote controlled) means.

According to another embodiment of invention, the aforesaid method further comprises a step of providing the device by a main longitudinal X axis; maneuvering at least a portion of said effecters along one or more of the X, Y, Z axes or any combination thereof, especially along the XY, XZ, YZ planes or any combination of those planes, such that one or more of said effecters are adapted to reciprocate along one or more of said axes, rotate around one or more of said axes, retract or otherwise expand or protract along one or more of said axes, maneuver upwards of downwards along one or more of said axes, set the effecters in their ON or OFF; Activated or Deactivated; Open (e.g., widen), gradually Close or completely Close (e.g., tighten); Loose or Secured modes or any combination thereof.

According to another embodiment of invention, the aforesaid method further comprises a step of providing said device with a length of the device is about 40 to about 60 cm, especially 50 cm; the outer width is between about 20 to about 50 mm, especially 25, 35 or 40 mm; the number of internal channels is between about 1 to about 10, especially 4, wherein 1^{st} channel is of about 5 to about 15 mm, especially 10 mm, useful for needles and suturing materials, or morcellators, 2^{nd} and 3^{rd} channels are of about 20 2 to about 10 mm, especially about 5 mm, useful for various instruments and fluids flow, and 4^{th} channel is of about 1 to about 5 mm, especially about 3 mm, useful for fluids flow, especially about for spillage and suction.

According to another embodiment of invention, the aforesaid method further comprises a step of providing said device by biocompatible building materials; and selecting at said materials from a group consisting of metals, especially stainless steel, polymers, shape memory alloys, especially nitinol, electrocative polymers, glassware, composite materials, cardboard or any mixture thereof.

According to another embodiment of invention, the aforesaid method further comprises a step of providing said proximal portion to be at least temporarily immobilized to a fixation, especially wherein said fixation is selected from the operation table or a moveable tripod.

According to another embodiment of invention, the aforesaid method of treating human body by the method as defined above; wherein said treatment is useful for applications selected from a group consisting (i) urological applications, especially nephrectomy, excision of ureteric and/or bladder stones, bladder tumors, kidney stones, kidney tumors, operation of the ureter, excisions biopsies and treatment thereof, treatment of stress incontinence through suturing, slings and/or mashes; (ii) surgical application, especially cholecystectomy, liver biopsies, gastric tumors splenectomy, gastrointestinal tumors (cholectomies), appendectomy, tumors of the pancreas (carcinoma, or insulinoma), or lymphydenectomy; (iii) endocrinological applications, especially suprarenal tumors or insuloma; (iv) gynecological applications: TED assisted hysterectomy (supracervical or total), TED total hysterectomy, 10 myomectomies, or treatment of fibroids with RF, cryotherapy, occlusion of blood vessels, excision of extrauterine pregmensies, tubal surgery; (v) cardiovascular applications, especially diagnostic or surgical, implantation of heart valves and grafts of or to the major blood vessels, operations on blood vessels, like the A. Renalis; and (vi) neurological applications: application of electrodes to nerves arising from the Sacral and Lumbar Plexi.

It is another object of the present invention to disclose a method of utilizing trans-Douglas endoscopic surgical procedures in the abdominal cavity and other body portions, especially in the gallbladder, blood vessels, nerves, liver, pancreas, spleen, kidney, colon, jejunum, or ileum, heart, nerve or combination thereof in human patient or otherwise female mammals. The method comprises steps of: opening the Pouch of Douglas; inserting an S-shapeable endoscopic surgical device into the vagina; possibly after insertion of a sleeve, introducing the distal end into the abdominal cavity, S-shaping the tubular assembly, and carrying out an operation in the abdominal cavity.

According to another embodiment of invention, the aforesaid method which is utilized for performing minimally invasive endoscopic intraabdominal procedures. The method further comprises steps of (I) providing an endoscopic surgical device being an elongated member and (II) providing said endoscopic surgical device with a distal portion located within said body cavity, comprising operating modules, and a proximal portion comprising controlling means handled by the surgeon, located outside said body cavity, and a middle portion, adapted to the pelvic shape, interconnecting said distal portion with said proximal portion; wherein said method further comprising steps of: a. providing said distal portion comprising either (i) one operating module (effecter), or (ii) two or more effecters, adapted to directly translate the surgeon's hands movement and orientation, as they interact with the tools; providing said effecters with means adapted to allow a single instrument to carry out procedures which normally would require both hands of the surgeon and/or two invasive endoscopic devices; providing said distal portion with auxiliary means, and selecting said auxiliary means from a group consisting of scopes or other image and data acquiring means, channels for tools, effecters and fluids inlets and/or outlet, lighting means, radiofrequency (RF), laser or ultrasound emitting means, sensors, diagnostic tools, OCT-diagnostic tools, optics, or any combination thereof; b. providing said proximal portion comprising controlling means adapted to maneuver and operate at least a portion of said effecters and auxiliary means; and, c. providing said middle portion interconnects said proximal portion with said distal portion, suitable to provide a predetermined shape, especially a shape selected from a shape adapted to the pelvic anatomy, namely U-like shape, S-like shape, partially linear shape and partially non-linear shape; said middle portion is preferably characterized by a tubular or polygonal cross section, and comprises a plurality of channels exceeded from the proximal end to the distal end.

According to another embodiment of invention, the aforesaid method further comprises a step of providing said device with configuration switching mechanism which is operated mechanically, hydraulically, electrically or by a combination of the same; said switching mechanism is possibly operated by either wired or wireless (remote controlled) means.

According to another embodiment of invention, the aforesaid method further comprises a step of providing said middle portion with either S-like or U-like shapes, adapted to the contour of the pelvis of the patient.

1. A method of providing trans-Douglas endoscopic surgical procedures in the pelvis and in other body portions, and especially gynecological, cardiological, urological, vascular, neurological or surgical operations; said method comprising steps of:
a. opening the Pouch of Douglas;
b. inserting an U-shapeable endoscopic surgical device into the vagina;
c. introducing the distal end into the abdominal cavity;
d. U-shaping the tubular assembly; and,
e. carrying out an operation in the pelvis.

2. The method according to claim 1 for performing minimally invasive endoscopic intraabdominal procedures; said method further comprising steps of (I) providing an endoscopic surgical device being an elongated member and (II) providing said endoscopic surgical device with a distal portion located within said body cavity, comprising operating modules, and a proximal portion comprising controlling means handled by the surgeon, located outside said body cavity, and a middle portion, adapted to the pelvic shape, interconnecting said distal portion with said proximal portion; wherein said method further comprising steps of:
a. providing said distal portion comprising either (i) one operating module (effecter), or (ii) two or more effecters, adapted to directly translate the surgeon's hands movement and orientation, as they interact with the tools; providing said effecters with means adapted to allow a single instrument to carry out procedures which normally would require both hands of the surgeon and/or two invasive endoscopic devices; providing said distal portion with auxiliary means, and selecting said auxiliary means from a group consisting of scopes or other image and data acquiring means, channels for tools, effecters and fluids inlets and/or outlet, lighting means, radiofiequency (RF), laser or ultrasound emitting means, sensors, diagnostic tools, OCT-diagnostic tools, optics, or any combination thereof;
b. providing said proximal portion comprising controlling means adapted to maneuver and operate at least a portion of said effecters and auxiliary means; and,
c. providing said middle portion interconnects said proximal portion with said distal portion: suitable to provide a predetermined shape, especially a shape selected from a shape adapted to the pelvic anatomy, namely U-like shape, S-like shape, partially linear shape and partially non-linear shape; said middle portion is preferably characterized by a tubular or polygonal cross section, and comprises a plurality of channels exceeded from the proximal end to the distal end.

3. The method according to claim 1; further comprising a step of providing said middle portion with either S-like or U-like shapes, adapted to the contour of the pelvis of the patient.

4. The method according to claim 1; further comprising a step of providing said device with an optic system, adapted to provide either 2D or 3D vision at or adjacent its distal end

5. The method according to claim 1; further comprising a step of providing said device configuration switching mechanism to either reversibly or irreversibly switching between two or more configurations, especially from initial configuration of approximately linear shape to either U-like or S-like shapes, and vice versa, from initial either U-like or S-like shapes to an approximated linear shape.

6. The method according to claim 1; further comprising a step of providing said device with configuration switching mechanism which is operated mechanically, hydraulically, electrically or by a combination of the same; said switching mechanism is possibly operated by either wired or wireless (remote controlled) means.

7. The method according to claim 1, especially useful for teaching and training purposes and/or operating multiple tasks simultaneously, wherein said proximal portion comprises a plurality of N sets of control handles, N is an integer number equal to or higher than 1, especially wherein N equals 2.

8. The method according to claim 1; further comprising a step of selecting at least a portion of said effecters from a group consisting of scissors, forceps, harmonic knife, monopolar knife, clips, blades, RF, cryotherapy, laser, monopolar knife and/or bipolar coagulation, ultrasound, spillage and suction modules, optics, especially optical means for 2D or 3D sight, light sources, loops and snare-like members, basket-like members, cleansing modules, and optical cleansing means.

9. The method according to claim 1; further comprising a step of selecting said optics from a group consisting of one or more either 2D or 3D scopes, one or more optic fibers, array of optic fibers, binocular, optical paths, imaging means, and especially OCT, ultrasound probes adapted for detection of blood vessels, tool for diagnostics or any combination of the same.

10. The method according to claim 1 further comprising a step of providing the device with optical cleansing modules, and providing the device with means for applying fluids, especially saline towards the optic tip portion located at the distal portion of the device; and especially wherein said cleansing modules and optical cleansing means are applied as high pressure water sprinkles directed to at least one scope distal tip.

11. The method according to claim 10; further comprising a step of providing said cleansing modules with wiper adapted to wipe the optic tip.

12. The method according to claim 1; further comprising a step of operating at least a portion of said effecters by electrically, hydraulically, mechanically, robotically, or by any combination of those techniques; and manipulating said electrical operated effecters by either wired or wireless (remote controlled) means.

13. The method according to claim 1; further comprising a step of providing the device by a main longitudinal X axis; maneuvering at least a portion of said effecters along one or more of the X, Y, Z axes or any combination thereof, especially along the XY, XZ, YZ planes or any combination of those planes, such that one or more of said effecters are adapted to reciprocate along one or more of said axes, rotate around one or more of said axes, retract or otherwise expand or protract along one or more of said axes, maneuver upwards of downwards along one or more of said axes, set the effecters in their ON or OFF; Activated or Deactivated; Open (e.g., widen), gradually Close or completely Close (e.g., tighten); Loose or Secured modes or any combination thereof.

14. The method according to claim 1; further comprising a step of providing said device with a length of the device is about 40 to about 60 cm, especially 50 cm; the outer width is between about 20 to about 50 mm, especially 25, 35 or 40 mm; the number of internal channels is between about 1 to about 10, especially 4, wherein 1^{st} channel is of about 5 to about 15 mm, especially 10 mm, useful for needles and suturing materials, or morcellators, 2^{nd} and 3^{rd} channels are of about 20 2 to about 10 mm, especially about 5 mm, useful for various instruments and fluids flow, and 4^{th} channel is of about 1 to about 5 mm, especially about 3 mm, useful for fluids flow, especially about for spillage and suction.

15. The method according to claim 1; further comprising a step of providing said device by biocompatible building materials; and selecting at said materials from a group consisting of metals, especially stainless steel, polymers, shape memory alloys, especially nitinol, electrocative polymers, glassware, composite materials, cardboard or any mixture thereof.

16. The method according to claim 1; further comprising a step of providing said proximal portion to be at least temporarily immobilized to a fixation, especially wherein said fixation is selected from the operation table or a moveable tripod.

17. A method of treating human body by the method as defined in claim 1; wherein said treatment is useful for applications selected from a group consisting (i) urological applications, especially nephrectomy, excision of ureteric and/or bladder stones, bladder tumors, kidney stones, kidney tumors, operation of the ureter, excisions biopsies and treatment thereof, treatment of stress incontinence through suturing, slings and/or mashes; (ii) surgical application, especially cholecystectomy, liver biopsies, gastric tumors splenectomy, gastrointestinal tumors (cholectomies), appendicectonmy, tumors of the pancreas (carcinoma, or insulinoma), or lymphydenectomy; (iii) endocrinological applications, especially suprarenal tumors or insulinoma; (iv) gynecological applications: TED assisted hysterectomy (supracervical or total), TED total hysterectomy, 10 myomectomies, or treatment of fibroids with RF, cryotherapy, occlusion of blood vessels, excision of extrauterine pregmensies, tubal surgery; (v) cardiovascular applications, especially diagnostic or surgical, implantation of heart valves and grafts of or to the major blood vessels, operations on blood vessels, like the A. Renalis; and (vi) neurological applications: application of electrodes to nerves arising from the Sacral and Lumbar Plexi.

18. A method of providing trans-Douglas endoscopic surgical procedures in the abdominal cavity and other body portions, especially in the gallbladder, blood vessels, nerves, liver, pancreas, spleen, kidney, colon, jejunum, or ileum, heart, nerve or combination thereof; said method comprising steps of: opening the Pouch of Douglas; inserting an S-shapeable endoscopic surgical device into the vagina; possibly after insertion of a sleeve, introducing the distal end into the abdominal cavity, S-shaping the tubular assembly, and carrying out an operation in the abdominal cavity.

19. The method according to claim 18 for performing minimally invasive endoscopic intraabdominal procedures; said method further comprising steps of (I) providing an endoscopic surgical device being an elongated member and (II) providing said endoscopic surgical device with a distal portion located within said body cavity, comprising operating modules, and a proximal portion comprising controlling means handled by the surgeon, located outside said body cavity, and a middle portion, adapted to the pelvic shape, interconnecting said distal portion with said proximal portion; wherein said method further comprising steps of:
a. providing said distal portion comprising either (i) one operating module (effecter), or (ii) two or more effecters, adapted to directly translate the surgeon's hands movement and orientation, as they interact with the tools; providing said effecters with means adapted to allow a single instrument to carry out procedures which normally would require both hands of the surgeon and/or two invasive endoscopic devices; providing said distal portion with auxiliary means, and selecting said auxiliary means from a group consisting of scopes or other image and data acquiring means, channels for tools, effecters and fluids inlets and/or outlet, lighting means, radiofrequency (RF), laser or ultrasound emitting means, sensors, diagnostic tools, OCT-diagnostic tools, optics, or any combination thereof;
b. providing said proximal portion comprising controlling means adapted to maneuver and operate at least a portion of said effecters and auxiliary means; and,
c. providing said middle portion interconnects said proximal portion with said distal portion; suitable to provide a predetermined shape, especially a shape selected from a shape adapted to the pelvic anatomy, namely U-like shape, S-like shape, partially linear shape and partially non-linear shape; said middle portion is preferably characterized by a tubular or polygonal cross section, and comprises a plurality of channels exceeded from the proximal end to the distal end.

20. The method according to claim 18; further comprising a step of providing said device with configuration switching mechanism which is operated mechanically, hydraulically, electrically or by a combination of the same; said switching mechanism is possibly operated by either wired or wireless (remote controlled) means.

21. The method according to claim 18; further comprising a step of providing said middle portion with either S-like or U-like shapes, adapted to the contour of the pelvis of the patient.

22. An endoscopic surgical device for use in performing minimally invasive endoscopic intraabdominal procedures; said device is an elongated member and characterized by a distal portion located within said body cavity, comprising operating modules, and a proximal portion comprising controlling means handled by the surgeon, located outside said body cavity, and a middle portion, adapted to the pelvic shape, interconnecting said distal portion with said proximal portion; wherein
a. said distal portion comprising either (i) one operating module (effecter), or (ii) two or more effecters, adapted to directly translate the surgeon's hands movement and orientation, as they interact with the tools; said effecters allow a single instrument to carry out procedures which normally would require both hands of the surgeon and/or two invasive endoscopic devices; said distal portion further comprising auxiliary means, selected from a group consisting of scopes or other image and data acquiring means, channels for tools, effecters and fluids inlets and/or outlet, lighting means, radiofrequency (RF), laser or ultrasound emitting means, sensors, diagnostic tools, OCT-diagnostic tools, optics, or any combination thereof;
b. said proximal portion comprising controlling means adapted to maneuver and operate at least a portion of said effecters and auxiliary means;
c. said middle portion interconnects said proximal portion with said distal portion; suitable to provide a predetermined shape, especially a shape selected from a shape adapted to the pelvic anatomy, namely U-like shape. S-like shape, partially linear shape and partially non-linear shape; said middle portion is preferably characterized by a tubular or polygonal cross section, and comprises a plurality of channels exceeded from the proximal end to the distal end.

23. The endoscopic surgical device according to claim 22, wherein said middle portion is characterized by either S-like or U-like shapes, adapted to the contour of the pelvis of the patient.

24. The endoscopic surgical device according to claim 22, wherein said middle portion is adapted to either reversibly or irreversibly switch between two or more configurations, especially from initial configuration of approximately linear shape to either U-like or S-like shapes, and vice versa, from initial either U-like or S-like shapes to an approximated linear shape.

25. The endoscopic surgical device according to claim 24, wherein said configuration switching mechanism is operated mechanically, hydraulically, electrically or by a combination of the same; said switching mechanism is possibly operated by either wired or wireless (remote controlled) means.
